(19) United States
(12) Patent Application Publication
(10) Pub. No.: US 2026/0240620 A1
(43) Pub. Date: Aug. 20, 2026

(54) DEVICE FOR STORING INSTRUMENTS OR IMPLANTS ON AN INSTRUMENT TRAY

(71) Applicant: Aesculap AG, Tuttlingen (DE)

(72) Inventor: Bianca Rosin, Emmingen-Liptingen (DE)

(21) Appl. No.: 19/537,799

(22) Filed: Feb. 12, 2026

(30) Foreign Application Priority Data
Feb. 20, 2025 (DE) 10 2025 106 555.1

(51) Int. Cl. A61B 50/33

(52) U.S. Cl. CPC A61B 50/33

(57) ABSTRACT

A device is used for storing one or more medical instruments and/or implants on an instrument tray. The device has a tray support structure and instrument storing part with a base portion that can be placed against the tray support structure. A wall portion rises from the base portion to receive instruments and/or implants. A fastening element releasably fixes the instrument storing part to the tray support structure. The fastening element has a flexible tongue extending toward the tray support structure. A limb is supportable against the base portion. A cutout is adapted to the outer contour of the fastening element and extends through the base portion. The cutout is formed such that the fastening element and flexible tongue are insertable in the cutout. The fastening element is supported by the support limb against a side of the base portion and secured to the instrument tray.

### CROSS-REFERENCE TO RELATED APPLICATION

This application claims priority under 35 U.S.C. § 119 to German Application No. 10 2025 106 555.1, filed on Feb. 20, 2025, the content of which is incorporated by reference herein in its entirety.

### FIELD

The present disclosure relates to a device for storing and preferably holding one or more medical, in particular surgical, instruments and/or implants in an intended arrangement on an instrument tray for use in supplying sterile goods in the medical field, the device having a tray support structure having a plurality of tray openings, wherein the device comprises an instrument storing part, in particular a metal one, having a base portion that can be placed against the tray support structure and having at least one wall portion rising from the base portion for receiving the instruments and/or implants, and a fastening element for releasably fixing the instrument storing part to the tray support structure of the instrument tray. Furthermore, the present disclosure relates to an instrument tray for use in supplying sterile goods in the medical field.

### BACKGROUND

Instrument trays, which are often designed as tray baskets or tray inserts for sterile goods containers, as well as the aforementioned devices for storing and holding instruments and/or implants in such instrument trays, play an important role in the so-called sterile goods cycle of medical and, in particular, surgical instruments and, in some cases, implants. They receive the instruments to be sterilized during sterilization and also afterward during transport to the sterile goods storage facility and from there to the place of use, in particular an operating room. For this purpose, numerous so-called organization systems for instrument trays of the type mentioned above have already been proposed for keeping the instruments in a predetermined position and arrangement on the instrument trays until they are removed by medical personnel immediately before they are used. Previously known devices for storing one or more instruments on the instrument tray can either be fastened to the instrument tray or its tray support structure by means of a screw connection, which is considered to be complex, or are inserted through the tray support structure from the lower side of the tray support structure through tray openings or other specially designed openings such that they protrude upward on the top side of the instrument tray in order to be able to store instruments there. Both imply that the retaining device is to some extent assembled on both sides of the instrument tray. The storage or retaining devices can therefore only be configured on the tray support structure before instruments are actually being loaded, since the instrument tray must be tilted and turned for this purpose. Mounting elements arranged on the lower side of the instrument tray have also proven to be disruptive; in particular, they can lead to abrasion, damage to the support surface or, if not distributed uniformly, to the instrument tray wobbling on the support surface.

For example, there are metal instrument storing parts that are manufactured from a strip portion of sheet metal by means of a few bending/punching cuts. They have a substantially planar, strip-shaped base portion and wall portions rising from the ends thereof, which, in particular through V-shaped shaping or in some other way, have storing points for receiving an instrument or implant. These instrument storing parts are typically screwed onto the tray support structure of an instrument tray using screws and nuts, which is considered to be a laborious process. Firstly, an auxiliary tool is required for assembly. Secondly, the instrument tray must be accessed from above and below. A screw must be inserted from below through a tray opening and held in place, and furthermore the strip-shaped base portion of the instrument storing part must be held against rotating while a nut is simultaneously being tightened with a tool.

### SUMMARY

Based on this, the object of the present disclosure aims to make the releasable arrangement of a device of the type mentioned at the outset for storing one or more instruments or implants on an instrument tray more user-friendly, wherein the releasable fastening of the device to the tray support structure should be possible without tools.

This object is achieved according to the present disclosure by a device of the type mentioned at the outset in that the fastening element has at least one flexible tongue that extends toward the tray support structure of the instrument tray and that can be locked thereto, and at least one support limb that can be supported against the base portion of the instrument storing part, and in that a cutout that is adapted to the outer contour of the fastening element and extends through the base portion is formed in the base portion of the instrument storing part, such that the fastening element together with the flexible tongue thereof can be inserted in the cutout, such that the flexible tongue of the fastening element can be locked to the tray support structure and the fastening element is then supported by the support limb thereof against a side of the base portion of the instrument storing part facing away from the tray support structure, such that the instrument storing part is thereby secured to the instrument tray.

It is therefore proposed according to the present disclosure that the fastening element for the actual instrument storing part itself can be locked to the tray support structure of an instrument tray by means of at least one flexible tongue and, in this way, can be secured to the tray support structure together with the actual instrument storing part. For this purpose, the aforementioned cutout is provided in the base portion of the instrument storing part, into which cutout the fastening element together with the flexible tongue thereof can be inserted, such that the flexible tongue can engage through this cutout into the tray support structure below. The base portion of the instrument storing part is thus arranged between the tray support structure and the fastening element, wherein the fastening element is supported by the at least one support limb thereof against the base portion and thereby exerts a clamping force on the base portion. This ensures that the instrument storing part is held without play against the tray support structure of the instrument tray. It is considered preferable for the fastening element to have two opposing support limbs, since this allows a uniform clamping pressure to be exerted on the base portion of the instrument storing part at two locations. It is also conceivable and advantageous if the base portion of the instrument storing part has a plurality of cutouts for a corresponding plurality of fastening elements for the one instrument storing part. This allows even an elongate, protruding instrument storing part to be securely and releasably secured to the instrument tray. The releasable fixing of the fastening element itself to the tray support structure by means of the flexible tongue thereof can be carried out as already described in DE 10 2023 103 493 A1. The disclosure of this publication in this regard is included in the present application.

It has further proven particularly advantageous if the support limb of the fastening element is designed to be flexible, such that different thicknesses of the base portion of the instrument storing part can be accommodated without play. In this way, one and the same fastening element can be used for different instrument storing parts with base portions of varying thickness.

It is further proposed that the cutout in the base portion of the instrument storing part is assigned a further, in particular slot-shaped, cutout or depression and is formed in the base portion, into which cutout or depression the support limb of the fastening element can engage by the free end thereof. Allowing the support limb to engage in this further, in particular slot-shaped, cutout or depression in the base portion of the instrument storing part assists with the intended arrangement and assembly of the fastening element and instrument storing part on the tray support structure, and unintentional rotation of the fastening element relative to the base portion is prevented. Preferably, each cutout is assigned two such slot-shaped cutouts or depressions on opposite sides for receiving support limbs of the fastening element on both sides.

It has further proven advantageous if the support limb extends obliquely upward through the cutout in the base portion of the instrument storing part, proceeding from a lower region of the fastening element which faces the instrument tray and which can be inserted in this cutout, beyond the base portion and then angled back toward the base portion. While the flexible tongues extend downward to lock in the tray support structure, the support limb extends in the opposite direction, first obliquely upward with a limb portion extending obliquely upward and then downward again with a limb portion angled downward to rest against the base portion of the instrument storing part. This allows a clamping force directed downward, i.e., toward the instrument tray, and in particular a clamping force generated by spring preload to be exerted on the base portion of the instrument storing part.

It is conceivable that the wall portion of the instrument storing part is designed for directly or indirectly receiving the instruments and/or implants. For this purpose, it may be widened at the upper end approximately in a V-shape or designed for attaching or slipping on an additional holder or molded part.

It is further advantageous if the fastening element is a sheet metal bent part and the flexible tongue and the support limb are notched out of the sheet metal bent part, i.e., each is formed from the material of the sheet metal bent part itself.

It is further proposed that the fastening element has a plurality of flexible tongues, preferably arranged opposite one another, i.e., deflectable in opposite directions, in particular one or two flexible tongues per side, and/or has a plurality of support limbs which are preferably arranged opposite one another. One embodiment of the fastening element that has proven to be preferable has two flexible tongues that are opposite one another and can be deflected in opposite directions, and two support limbs arranged opposite one another; furthermore, an embodiment which has two flexible tongues on each side, i.e., two times two flexible tongues opposite one another, and again two support limbs opposite one another.

In particular, when the fastening element is designed as a sheet metal bent part, it has proven advantageous if the fastening element has a main limb that can be placed against the top side of the tray support structure and two opposing side limbs adjoining it laterally.

In particular, it has proven advantageous if the opposing side limbs of the fastening element delimit an upwardly open U-shape, which is, however, somewhat narrowed toward the top by the side limbs being inclined toward one another and, in particular, being able to have a fold at the upper end.

It is advantageous that at least one flexible tongue is formed proceeding from one or both side limbs of the fastening element and extends downward from the side limbs of the fastening element toward the tray support structure.

In this case, the flexible tongue in the initial state of the fastening element can be pre-assembled in such a way that it forms an acute angle with the side limb and is bent outward with respect to the side limb, in particular by 5° to 25°.

It has further proven advantageous that the fastening element can be inserted in the cutout in the base portion of the instrument storing part, such that the fastening element can be positioned by the main limb thereof on the top side of the tray support structure, and that then, simply by applying a pressure force to the fastening element in the direction of the top side of the tray support structure, the flexible tongue is deflected and thereby automatically penetrates a tray opening and locks with an edge region of the tray support structure that delimits the tray opening, such that the fastening element and thus the instrument storing part can be attached to the tray support structure without tools and is captive for the intended use. When the fastening element inserted into the cutout in the base portion is pressed down or pushed in, the flexible tongue or tongues run against an edge region of the tray support structure that defines a tray opening; it is consequently deflected outward with respect to the fastening element and then latches in a rear-engagement situation, in that the flexible tongue springs back after engaging in a tray opening and thereby engages an edge region of the tray support structure that delimits the tray opening.

In a further development of this idea, it has proven advantageous that a free end of the flexible tongue is designed or shaped to form a rear-engaging region or rear-engaging bead, and that a free end of the flexible tongue is angled by more than 90°, preferably by at least 100°, preferably by at least 110° to the longitudinal extent of the flexible tongue, thus forming a rear engagement with an edge region of the tray support structure that delimits a tray opening.

In this respect, it has also proven advantageous that a free end of the flexible tongue forms a run-on slope which, when the fastening element is pressed in the direction of the top side of the tray support structure, runs against an edge region of the tray support structure delimiting a tray opening and is thereby deflected in order to latch with the edge region in a rear-engagement position.

In conjunction with a three-dimensionally corrugated tray support structure or a tray support structure having egg-carton-like raised portions and depressions, it has proven advantageous if recesses are formed in the fastening element, in particular in the main limb of the fastening element, into which recesses the tray support structure can engage with a raised portion from below, such that further position stabilization of the fastening element on the tray support structure can be achieved.

It is further proposed that the instrument tray has a tray support structure having in particular rectangular and preferably square tray openings which are delimited by connecting pieces that are narrower than the side length of the rectangular shape or than the largest dimension of the tray openings.

It has also proven advantageous that the instrument tray is rectangular in the top view of the top side, with a corresponding longitudinal direction and transverse direction, and that the connecting pieces delimiting the tray openings extend at an inclination of 45° to the longitudinal direction or transverse direction.

Furthermore, it has proven advantageous that the instrument tray comprises a three-dimensionally structured, in particular corrugated, tray support structure, and in particular that the fastening element can be arranged and fastened in the region of high points, raised portions or bumps of the tray support structure.

It has also proven advantageous that a distance between two adjacent flexible tongues of the fastening element corresponds to a distance between two intersection points of connecting pieces of the tray support structure that delimit the tray openings, or is twice or an integer multiple thereof, such that the flexible tongues can be automatically positioned in respective corner regions of the tray openings.

In an instrument tray according to the present disclosure having a retaining device for instruments according to the present disclosure, it has proven advantageous if a release instrument is provided which can be applied from the inside against one or more flexible tongues of the fastening element and thereby deflects said flexible tongue out of the rear-engagement position thereof, such that the fastening element can be released from rear-engagement position thereof in the instrument tray.

The instrument tray can advantageously be used as an instrument tray basket or as an insert for an instrument tray basket or instrument container.

### BRIEF DESCRIPTION OF THE DRAWINGS

Further features, details and advantages of the present disclosure can be found in the drawings and the following description.

In the drawings:

FIG. 1 shows a perspective exploded depiction of an instrument tray according to the present disclosure having a tray support structure and having two devices according to the present disclosure for storing an instrument (not depicted), each comprising an instrument storing part and a fastening element for releasably fixing the instrument storing part to the instrument tray;

FIG. 2 shows a perspective depiction of the instrument storing parts depicted in FIG. 1;

FIG. 3a shows a perspective depiction of a first embodiment of the fastening element depicted in FIG. 1 for releasably fixing the instrument storing part to the instrument tray;

FIG. 3b shows a perspective depiction of a second embodiment of the fastening element depicted in FIG. 1 for releasably fixing the instrument storing part to the instrument tray;

FIG. 4a shows a top view of a planar base portion of the instrument storing part having a cutout for receiving the fastening element according to FIG. 3a;

FIG. 4b shows a top view of a planar base portion of the instrument storing part having a cutout for receiving the fastening element according to FIG. 3b;

FIG. 5a shows a depiction corresponding to FIG. 4a with the fastening element according to FIG. 3a inserted into the cutout;

FIG. 5b shows a depiction corresponding to FIG. 4b with the fastening element according to FIG. 3b inserted into the cutout;

FIGS. 6a-6f show different views to illustrate the releasable securing of the fastening element to the tray support structure, for the sake of easier comprehension without the intermediate arrangement of an instrument storing part and without the fastening element having a support limb according to the present disclosure;

FIG. 7 shows a sectional view through a tray support structure with a device according to the present disclosure releasably locked thereto and depicted on the left in FIG. 1, for storing an instrument (not depicted) (the device having the instrument storing part depicted on the left in FIG. 1 and the fastening element depicted in FIG. 3b); and

FIG. 8 shows a perspective depiction of a release instrument for the fastening element.

### DETAILED DESCRIPTION

The drawings show an instrument tray 2 according to the present disclosure for medical, in particular surgical, instruments and/or implants, for use in supplying sterile goods in the medical field. The instrument tray 2 comprises a tray support structure 4 having a plurality of tray openings 6 and, by way of example, two devices 10 according to the present disclosure (depicted floating at the top) for storing and preferably for holding an instrument (not depicted in the drawings) during the sterile goods cycle. Each device 10 comprises an instrument storing part 12 having a base portion 14 that can be placed against the tray support structure 4 and two wall portions 16 rising from the base portion 14 for receiving the instruments and/or implants (not depicted). Furthermore, each device 10 comprises a fastening element 18 for releasably fixing the instrument storing part 12 to the instrument tray 2 or tray support structure 4, which will be explained below.

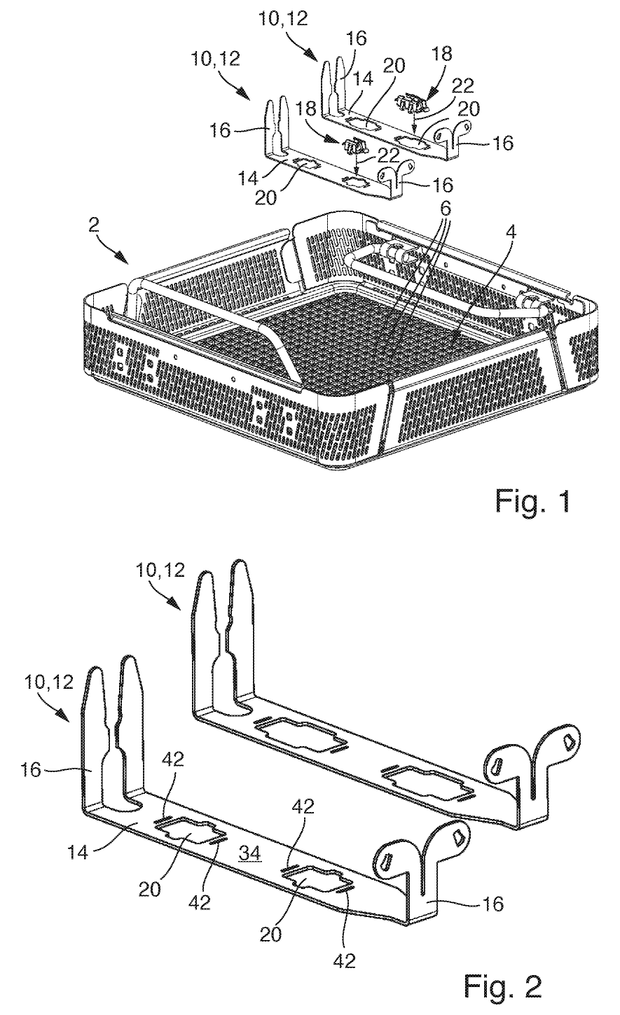

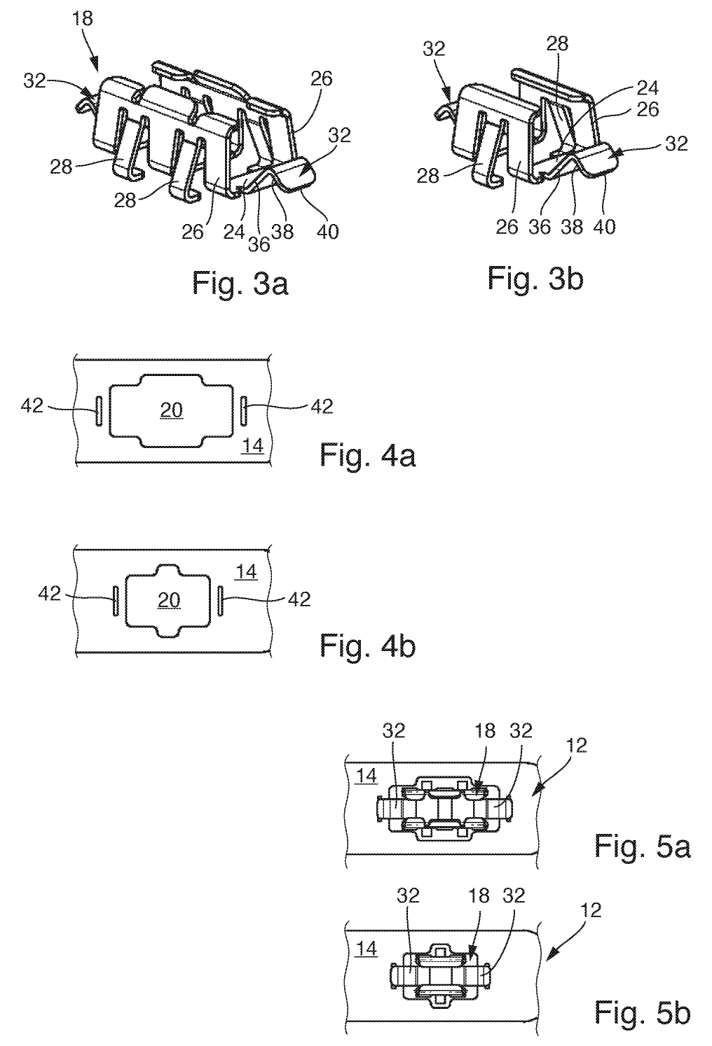

The instrument storing parts 12 and fastening elements 16 depicted in FIG. 1 are depicted enlarged in FIGS. 2 and 3a, 3b. Each instrument storing part 12 comprises a base portion 14 which is planar in the case depicted and, at the two longitudinal ends, comprises aforementioned wall portions 16 projecting orthogonally upward from the base portion 14 for receiving the instruments. In the base portion 14, a cutout 20 is formed which is adapted to the outer contour of the fastening element 18, such that the fastening element 18 can be inserted into this cutout 20 in the direction of the arrow 22 in FIG. 1 and can enter a positive locking connection with the tray support structure 4 of the instrument tray 2 in a manner to be explained below. In the process, the fastening element 18 exerts a clamping force on the base portion 14 of the instrument storing part 12 via, for example, two opposing support limbs 32 at the same time, and thus simultaneously secures the instrument storing part 12 against the tray support structure 4.

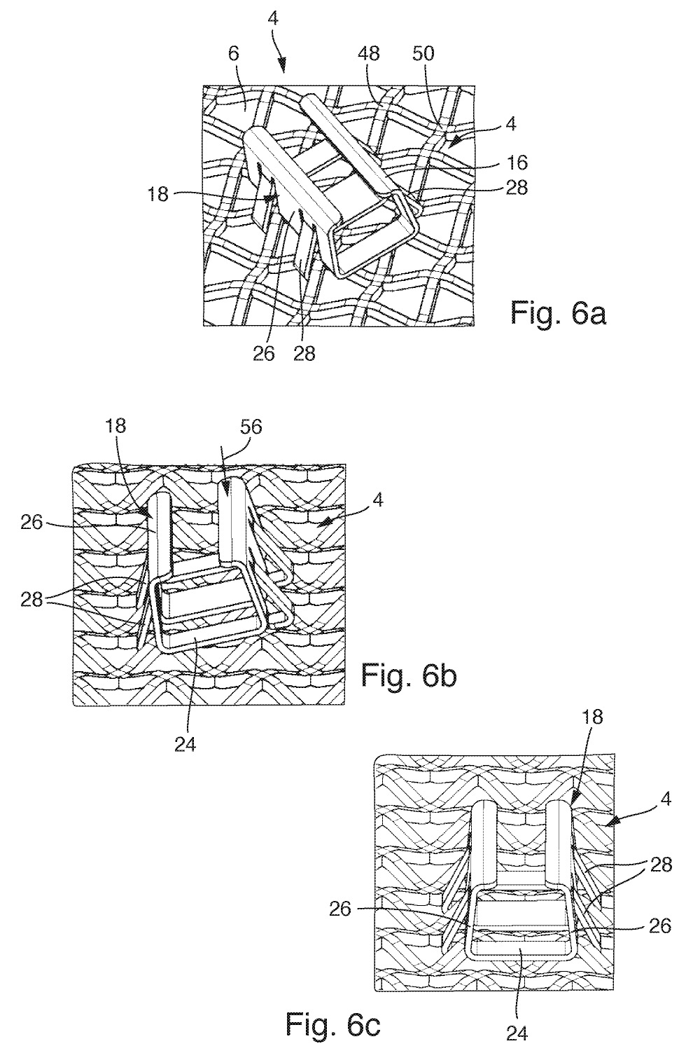

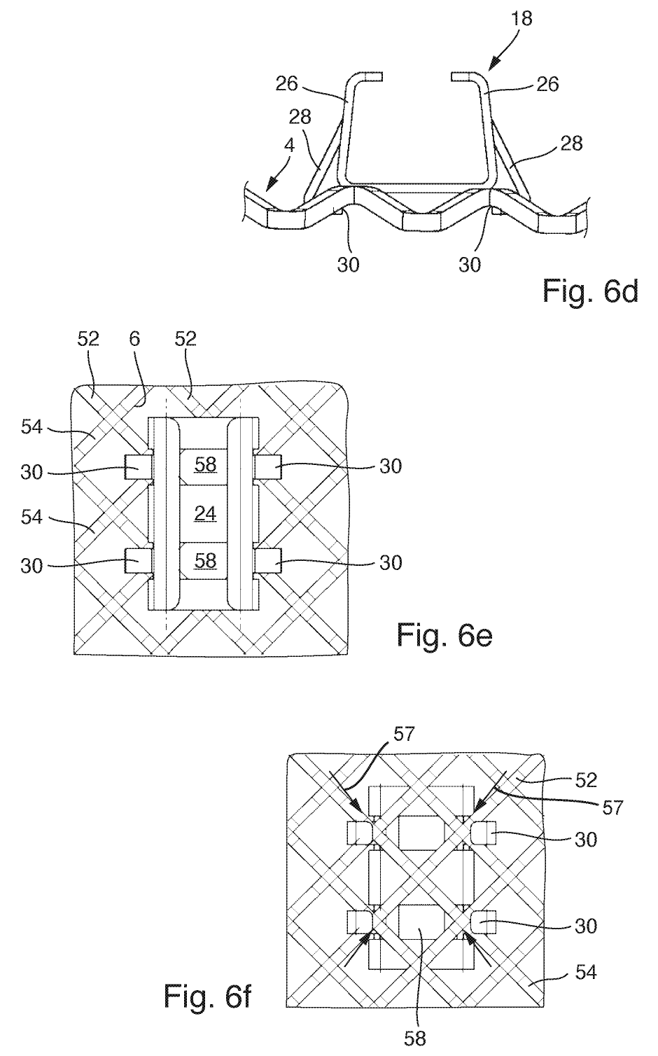

The fastening element 18 depicted in two embodiments in FIG. 3a and 3b is formed, by way of example, from a sheet metal bent part. In the exemplary embodiment, it comprises a main limb 24 and, adjoining it laterally, two opposing side limbs 26, such that the fastening element 18 forms an approximate U-shape. Proceeding from an upper region of the side limbs 26, two flexible tongues 28 are formed on each side limb according to FIG. 3a and extend from top to bottom, i.e., in the direction of the tray support structure 4. They have an angled, rear-engaging region 30 at their free end, such that they can be locked with the tray support structure 4 and the tray openings 6 thereof, and thus are able to hold and secure the fastening element 18 to the tray support structure 4 with the intermediate arrangement of the base portion 14 of the instrument storing part 12, which will be explained in detail below with reference to FIGS. 6a-f. Furthermore, in the case depicted by way of example, each fastening element 18 has a support limb 32 on opposite longitudinal sides, by means of which the fastening element 18 is then supported against an upper side 34 of the base portion 14 of the instrument storing part 12 facing away from the tray support structure 4. Each support limb 32 is designed to be flexible and can thus be used together with instrument storing parts 12 having base portions 14 of different thicknesses.

Each support limb 32 extends obliquely upward from a lower region of the fastening element 18, which engages in the cutout 20, and is then angled downward again. The support limb 32 thus comprises a limb portion 36 extending obliquely upward from the main limb 24 of the U-shape and subsequently a limb portion 38 angled downward. The support limb 32 presses by a free end 40 of said downwardly angled limb portion 38 onto the base portion 14 of the instrument storing part 12 and exerts the aforementioned clamping force on the base portion 14 of the instrument storing part 12. This allows the instrument storing part 12 to be releasably secured against the tray support structure 4.

In addition to the relevant cutout 20, further slot-shaped cutouts or depressions 42 are provided in the base portion 14 in the exemplary case (see FIGS. 1 and 4a, 4b), which form a positioning aid for the support limbs 32 or rather the free ends 40 thereof.

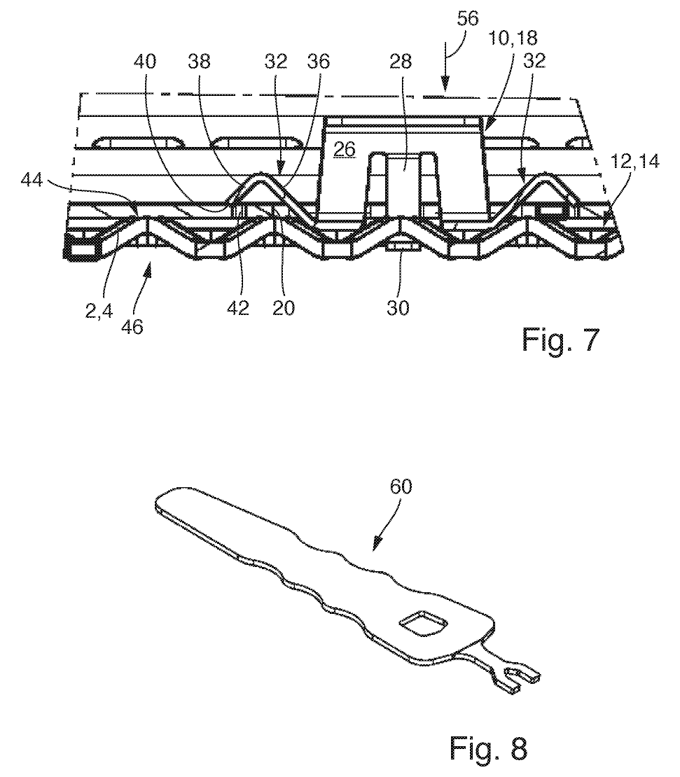

The fastening element 18 can be releasably fastened to the tray support structure 4 in a manner to be described below. For a clearer and more understandable depiction of the fastening, the instrument storing part 12 arranged between fastening element 18 and tray support structure 4 is not depicted in FIGS. 6a-f but rather omitted. Likewise, the support limb 32 according to the present disclosure has been omitted. A sectional depiction with the instrument storing part 12 plus support limb 32 interposed according to the present disclosure is depicted in FIG. 7. The tray support structure 4 in FIGS. 6a-f has a top side 44 facing the device 10 and the instruments (not depicted) and a lower side 46 facing away therefrom. In the case depicted by way of example here, the tray support structure 4 is corrugated in two directions and thus has raised portions 48 and depressions 50. The tray support structure 4 is formed, for example, from two groups each consisting of a plurality of connecting pieces 52, 54 that extend parallel to one another when projected onto the plane (FIGS. 6e, f), wherein the connecting pieces 52 of one group extend at right angles to the connecting pieces 54 of the other group, such that they delimit the tray openings 6. Proceeding from an initially planar tray support structure 4 consisting of the connecting pieces 52, 54, the structure having raised portions 48 and depressions 50 was then embossed in such a way that raised portions 48 and depressions 50 alternate in the direction of the connecting pieces 52, 54. Despite the three-dimensional structure, the tray openings 6 can, for example, be described in the present case as being substantially square, i.e., they have a square shape when projected vertically onto the base surface of the tray support structure 4 (see FIGS. 6e, f).

FIGS. 6a-f show the exemplary three-dimensionally corrugated tray support structure 4 and the fastening element 18 with the flexible tongues 28 thereof. It can be seen in FIG. 6a, b that the fastening element 18 is first slightly inclined with respect to a plane of the tray support structure 4, such that the flexible tongues 28 of the side limb 26 depicted on the left in FIGS. 6a, b engage in two adjacent tray openings 6 in the region of in each case one raised portion 48 or wave crest of the tray support structure 4 and hook with the connecting pieces 52, 54 by means of the relevant rear-engaging region 30 in the region of an intersection point. Proceeding from this assembly situation, only a pressure force in the direction of an arrow 56 depicted in FIG. 6b now needs to be exerted on the fastening element 18 or the other side limb 26 in the direction of the tray support structure 4 such that the opposite flexible tongues 28 run against the connecting pieces 53, 54 with their rear-engaging end regions 30 and are thereby deflected outward and finally reach the rear-engagement situation depicted in FIGS. 6c-f, in which the flexible tongues 28 spring back inward after engaging in two tray openings 6 and thereby assume a captive assembly position on the tray support structure 4. The rear-engaging end regions 30 of the flexible tongues 28 self-center in the direction of the arrows 57 in FIG. 6f against the intersection points of the connecting pieces 52, 54 of the tray openings 6. In this assembly position, the fastening element 18 to some extent rests with its main limb 20 in the region of raised portions 48 of the tray support structure 4 and is again arranged in parallel with the plane of the tray support structure 4. However, in the embodiment depicted by way of example, the fastening element 18 does not rest entirely on top of the high points of the raised portions 48 of the tray support structure 4, but rather the high points or peaks of the raised portions 48 dip slightly from below into recesses 58 in the region of the main limb 24 of the fastening element 18, which have resulted here from the disengagement of the flexible tongues 28.

FIG. 7 shows, in a sectional depiction, the releasable assembly of the device 10 according to the present disclosure, now with instrument storing part 12 and fastening element 18 on the corrugated tray support structure 4. As described at the beginning, the instrument storing part 12 is placed by the base portion 14 thereof on the tray support structure 4. Then, a relevant fastening element 18 is inserted into the relevant cutout 20 from above, such that its main limb 24 rests on the corrugated tray support structure 4. The fastening element 18 is then releasably locked to the tray support structure 4 as described above (without intermediate arrangement of the instrument storing part 12 based on FIGS. 6a-f). FIG. 7 also shows that the relevant rear-engaging region 30 at the free end of the relevant flexible tongue 28 engages under the tray support structure 4 in the region of a raised portion 48 and is hooked there in the intersection region of connecting pieces 52, 54, after the fastening element 18 has been pressed against the tray support structure 4 in the direction of the arrow 56. In this state, the two opposing support limbs 32 are subjected to a slight upward load and thus exert a clamping force on the base portion 14 of the instrument storing part 12 via the downwardly angled limb 38. They engage by their free ends 40 into the relevant slot-shaped cutout 42 in the base portion 14 and are thereby positioned. The clamping force introduced via the support limbs 32 onto the base portion 14 ensures that the instrument storing part 12 is releasably secured to the tray support structure 4 in a non-rotatable and captive manner.

To release the retaining device 10 from the tray support structure 4, the flexible tongues 28 can be accessed by means of a release instrument 60 depicted in FIG. 8 in order to deflect the flexible tongues 28 outward against their spring tension, such that they are released from their rear-engagement situation with the connecting pieces 52, 54 of the tray support structure 4 and the fastening element 18 can first be pivoted upward from the tray support structure 4 and then moved to the other side so that the flexible tongues 28 on the opposite side limb 26 are also released from their rear-engagement situation and the fastening element 18 can then be lifted upward from the tray support structure 4 and from the cutout 20 in the base portion 14 of the instrument storing part 12.

Alternatively, with appropriate design of the length and preload of the flexible tongues 26, it would also be conceivable, in principle, that the fastening element 18 could be released without tools from the rear-engagement position with the connecting pieces 52, 54 and then lifted upward.

1. A device for storing one or more medical, in particular surgical, instruments and/or implants in an intended arrangement on an instrument tray for use in supplying sterile goods in the medical field, the device having a tray support structure having a plurality of tray openings, wherein the device comprises an instrument storing part having a base portion that can be placed against the tray support structure and having at least one wall portion rising from the base portion for receiving the instruments and/or implants, and a fastening element for releasably fixing the instrument storing part to the tray support structure of the instrument tray,
wherein
the fastening element has at least one flexible tongue that extends toward the tray support structure of the instrument tray and that can be locked thereto, and at least one support limb that can be supported against the base portion of the instrument storing part,
and wherein a cutout that is adapted to the outer contour of the fastening element and extends through the base portion is formed in the base portion of the instrument storing part, such that the fastening element together with the flexible tongue thereof can be inserted in the cutout, such that the flexible tongue of the fastening element can be locked to the tray support structure and the fastening element is then supported by the support limb thereof against a side of the base portion of the instrument storing part facing away from the tray support structure, such that the instrument storing part is thereby secured to the instrument tray.

2. The device according to claim 1, wherein
the support limb of the fastening element is designed to be flexible, such that different thicknesses of the base portion of the instrument storing part can be accommodated without play.

3. The device according to claim 1, wherein
the cutout in the base portion of the instrument storing part is assigned a further cutout or depression and is formed in the base portion, into which cutout or depression the support limb of the fastening element can engage by the free end thereof.

4. The device according to claim 3, wherein the further cutout is slot-shaped.

5. The device according to claim 1, wherein
the support limb extends obliquely upward through the cutout in the base portion of the instrument storing part, proceeding from a lower region of the fastening element which faces the instrument tray and which can be inserted in this cutout, beyond the base portion and then angled back toward the base portion.

6. The device according to claim 1, wherein the wall portion of the instrument storing part is designed for directly or indirectly receiving the instruments and/or implants.

7. The device according to claim 1, wherein the fastening element is a sheet metal bent part and the flexible tongue and the support limb are notched out of the sheet metal bent part.

8. The device according to claim 1, wherein the fastening element has a plurality of flexible tongues which are opposite one another, i.e., deflectable in opposite directions, and/or has a plurality of support limbs.

9. The device according to claim 1, wherein the fastening element has a main limb that can be placed against the tray support structure and two opposing side limbs adjoining it laterally.

10. The device according to claim 9, wherein at least one flexible tongue is formed proceeding from one or both side limbs of the fastening element and extends downward from the side limbs of the fastening element toward the tray support structure, and forms an acute angle with the side limb and is bent outward with respect to the side limb.

11. The device according to claim 9, wherein
the fastening element can be inserted in the cutout in the base portion of the instrument storing part, such that the fastening element can be positioned by the main limb thereof on the top side of the tray support structure, and that then, simply by applying a pressure force to the fastening element in the direction of the top side of the tray support structure, the flexible tongue is deflected and thereby automatically penetrates a tray opening and locks with an edge region of the tray support structure that delimits the tray opening, such that the fastening element and thus the instrument storing part can be attached to the tray support structure without tools and is captive for the intended use.

12. The device according to claim 1, wherein a free end of the flexible tongue is designed or shaped to form a rear-engaging region or a rear-engaging bead.

13. The device according to claim 12, wherein a free end of the flexible tongue is angled by more than 90°, preferably by at least 100°, preferably by at least 110° to the longitudinal extent of the flexible tongue, thus forming a rear engagement with an edge region of the tray support structure that delimits a tray opening.

14. The device according to claim 1, wherein a free end of the flexible tongue forms a run-on slope which, when the fastening element is pressed in the direction of the top side of the tray support structure, runs against an edge region of the tray support structure delimiting a tray opening and is deflected in order to latch with the edge region into a rear-engagement position.

15. The device according to claim 1, wherein recesses are formed in the fastening element, into which recesses the tray support structure can engage with a raised portion from below, such that further position stabilization of the fastening element on the tray support structure can be achieved.

16. An instrument tray for medical, in particular surgical instruments and/or implants for use in supplying sterile goods in the medical field, in particular operating theaters, having a tray support structure having a plurality of tray openings, comprising a device according to claim 1.

17. The instrument tray according to claim 16, wherein the instrument tray comprises a three-dimensionally structured tray support structure, and wherein the fastening element can be arranged and fastened in the region of high points, raised portions or bumps of the tray support structure.

18. The instrument tray according to claim 17, wherein the instrument tray comprises a three-dimensionally structured, corrugated, tray support structure.
